(19) United States
(12) Patent Application Publication
(10) Pub. No.: US 2025/0255745 A1
(43) Pub. Date: Aug. 14, 2025

(54) PERSONALIZED-FIT NASAL DILATOR

(71) Applicant: Breathewave Technologies Inc., Edgewater, NJ (US)

(72) Inventors: David J. Axelrad, Closter, NJ (US); Michael A. Linn, Ridgewood, NJ (US); Geoffrey P. Waite, Campton, NH (US); Christopher A. Beaudreau, Parker, CO (US)

(21) Appl. No.: 19/051,958

(22) Filed: Feb. 12, 2025

Related U.S. Application Data
(60) Provisional application No. 63/552,836, filed on Feb. 13, 2024.

(51) Int. Cl. A61F 5/08

(52) U.S. Cl. CPC A61F 5/08

(57) ABSTRACT

A method for providing a personalized nasal dilator and the resulting personalized nasal dilator is provided having one or more inserts that include one or more varied configurations that fit nasal passage anatomical configurations specific to an individual user. The method involves acquiring data on the individual user's face and/or nasal structure, analyzing data for the individual user to evaluate the individual user's anatomical and structural needs, and manufacturing a nasal dilator that is personalized for the individual user wherein the nasal dilator is configured, shaped, and dimensioned for the individual user based on the evaluated anatomical and structural needs of the individual user. The resulting nasal dilator has the one or more nasal inserts are shaped, sized, and configured based anatomical information of nasal passage surface features derived through an analysis of anatomical information from individual user.

### CROSS-REFERENCE TO RELATED APPLICATION(S)

This application claims priority to, and the benefit of, co-pending U.S. Provisional Application 63/552,836, filed Feb. 13, 2024, for all subject matter common to both applications. The disclosure of said provisional application is hereby incorporated by reference in its entirety.

### FIELD OF THE INVENTION

The present invention relates to nasal dilators that are suitable for increasing airflow through a user's nasal passages. In particular, the present invention relates to an anatomically shaped nasal dilator that is personalized by being uniquely configured, shaped, and dimensioned for a specific individual user.

### BACKGROUND

Internal nasal dilators exist that have both solid-walled and latticed or net-walled construction. These tend to be hollow conical frustums with a right-circular form, i.e., circularly symmetric or some other mass-produced shape. The therapeutic function of the upper ring and upper portion of the wall, (which is typically, but not necessarily, smaller than the bottom ring) is to open and keep open the internal nasal valve while the bottom ring and lower portion of the wall open the external nasal valve. Both the two rings and the wall of the dilator also function to keep the dilator in place when inserted into the user's nostril, sometimes incorporating surface features such as ribs or bumps. These devices are typically fabricated of an elastic material, such as a medical grade polymer like a silicone rubber or elastomer, creating a restorative elastic spring force when distorted, such that when inserted into the nostril, they are somewhat accommodating of the nostril shape and exert a radial force to open, and resist collapse of, the nasal valves. In such existing devices, these forces can be determined based on the inherent elastic spring force of the simple hollow right circular, conical frustum form or some other mass-produced shape, without any consideration of the actual shape of the nasal passage, not the actual radial forces required for either or both of patency and comfort. Typically, such devices are offered in a limited number of size options, often three or four sizes, with both the ring diameters and the length of the device scaling appropriately.

However, this approach (technology, device, system, methodology, etc.) experiences some shortcomings. Specifically, since they do not accommodate the actual shape of the nostril of a particular user, they can exert a dilation force upon the user's nostril that may be too great in some areas (causing discomfort) and too small in other areas (providing limited benefit) when in use, resulting in suboptimal performance in both comfort and nasal patency.

### SUMMARY

There is a need for a nasal dilator that is personalized, such that it conforms more closely to the anatomical shape, location, and direction-dependent elastic modulus of an individual user's nose, thus enabling controlled distribution of dilation force around the circumference of the device at different depths of insertion and matching the anatomical requirements to deliver optimal or improved combination of patency and comfort. This personalized fit design is achieved by a personalization method and system that uses an analysis of the unique nasal profile of the user, derived from data acquired from the user (such as, e.g., from one or more scans or images of the user's face, exterior, and interior of nose and nostrils), from which a personalized fit is designed. The personalized fit device, thus designed, is manufactured by a programmable manufacturing system, such as a digitally controlled process including additive manufacturing. There are three portions of the personalized method and system: a scanning subsystem, a data transformation subsystem, and a manufacturing subsystem.

In accordance with embodiments of the present invention, a method for providing a personalized nasal dilator is provided. The method involves acquiring data on an individual user's face and/or nasal structure; analyzing the data to evaluate anatomical and structural needs for the individual user; and manufacturing a nasal dilator that is personalized for the individual user wherein the nasal dilator is configured, shaped, and dimensioned for the individual user based on the evaluated anatomical and structural needs of the individual user.

In accordance with aspects of the present invention, acquiring data on an individual user's face and/or nasal structure involves using image data to build a three-dimensional (3D) profile of an internal profile of the individual user's nostrils.

In accordance with aspects of the present invention, acquiring data on an individual user's face and/or nasal structure involves using video or photo photogrammetry of one or more of the user's face, exterior of nose and nostrils, and interior of nose and nostrils.

In accordance with aspects of the present invention, acquiring data on an individual user's face and/or nasal structure involves using 3D imaging techniques. In some such aspects, 3D imaging techniques comprise one or more of: LiDAR, depth or Time of Flight (TOF) cameras, infrared (IR) time of flight, structured infrared (IR) light patterns, and multiple cameras.

In accordance with aspects of the present invention, acquiring data on an individual user's face and/or nasal structure involves using one or more of X-ray, computed tomography (CT) systems, and ultrasound imaging.

In accordance with aspects of the present invention, acquiring data on an individual user's face and/or nasal structure involves using acoustic rhinometry.

In accordance with aspects of the present invention, acquiring data on an individual user's face and/or nasal structure further involves acquiring supplemental posed images or 3D scans of the user comprising one or more of: an image or 3D scan of the user wrinkling their nose, an image or 3D scan of the user flaring their nostrils, an image or 3D scan of the user forcefully inhaling through their nose, an image or 3D scan where the user holds one nostril closed and an image or 3D scan where the user holds other nostril closed; and an image or 3D scan where the user holds one nostril open and an image or 3D scan where the user holds other nostril open.

In accordance with aspects of the present invention, acquiring data on an individual user's face and/or nasal structure further involves extracting one or more landmarks.

In accordance with aspects of the present invention, acquiring data on an individual user's face and/or nasal structure comprises collecting user profile information.

In accordance with aspects of the present invention, analyzing data for the individual user to evaluate anatomical and structural needs for the individual user involves creating a dilator profile. In some such aspects, creating a dilator profile involves a geometric transformation from a 3D Nasal Profile for the user. In other such aspects, creating a dilator profile involves creating one or more circumferential radial force profiles.

In accordance with aspects of the present invention, analyzing data for the individual user to evaluate anatomical and structural needs for the individual user further involves receiving one or more pro-forma dilator designs, which comprise one or more of: stored templates, one or more stored rules, and one or more stored algorithms; and modifying the one or more pro-forma dilator designs according to data acquired.

In accordance with aspects of the present invention, analyzing data for the individual user provided to evaluate anatomical and structural needs for the individual user further involves receiving a 3D Nasal Profile for the user and modifying the 3D Nasal Profile for the user using one or more of: one or more stored templates, one or more stored rules, and one or more stored algorithms.

In accordance with aspects of the present invention, acquiring data on an individual user's face and/or nasal structure involves collecting and analyzing data about user response to a nasal dilator.

In accordance with aspects of the present invention, manufacturing a nasal dilator that is personalized for the individual user involves using additive manufacturing methods.

In accordance with aspects of the present invention, manufacturing a nasal dilator that is personalized for the individual user involves fabricating a mold for the personalized nasal dilator and using the mold to make a personalized nasal dilator.

In accordance with aspects of the present invention, manufacturing a nasal dilator that is personalized for the individual user involves modifying an existing dilator to personalize the dilator for the individual user.

In accordance with embodiments of the present invention, system for providing a personalized nasal dilator is provided. The system includes a data acquisition subsystem configured to receive data on an individual user's facial and nasal structures, a data transformation subsystem configured to process the data to evaluate nasal characteristics of the individual user; and a manufacturing subsystem configured to fabricate a customized fitting for the nasal dilator based on the evaluated nasal characteristics.

In accordance with aspects of the present invention, the data acquisition subsystem is configured to scan an individual user's face and/or nasal structure to build a three-dimensional (3D) profile of an internal profile of the individual user's nostrils.

In accordance with aspects of the present invention, the data acquisition subsystem is configured to use video or photo photogrammetry of one or more of a user's face, exterior of nose and nostrils, and interior of nose and nostrils.

In accordance with aspects of the present invention, the data acquisition subsystem is configured to use 3D imaging techniques.

In accordance with aspects of the present invention, the data acquisition subsystem is configured to use one or more of X-ray, computed tomography (CT) systems, and ultrasound imaging.

In accordance with aspects of the present invention, the data acquisition subsystem is configured to use acoustic rhinometry.

In accordance with aspects of the present invention, the data acquisition subsystem is configured to acquire supplemental posed images or 3D scans of the user including one or more of: an image or 3D scan of the user wrinkling their nose, an image or 3D scan of the user flaring their nostrils, an image or 3D scan of the user forcefully inhaling through their nose, an image or 3D scan where the user holds one nostril closed and an image or 3D scan where the user holds another nostril closed, and an image or 3D scan where the user holds one nostril open and an image or 3D scan where the user holds other nostril open.

In accordance with aspects of the present invention, the data acquisition subsystem is configured to extract one or more landmarks.

In accordance with aspects of the present invention, the data transformation subsystem is further configured to collect user profile information.

In accordance with aspects of the present invention, processing the data to evaluate nasal characteristics of the user comprises creating a dilator profile.

In accordance with aspects of the present invention, the data transformation subsystem is configured to receive one or more pro-forma dilator designs, which comprise one or more of: stored templates, one or more stored rules, and one or more stored algorithms and modify the one or more pro-forma dilator designs according to data provided by the acquisition.

In accordance with aspects of the present invention, the data transformation subsystem is configured to receive a 3D Nasal Profile for the user and modify the 3D Nasal Profile for the user using one or more of: one or more stored templates, one or more stored rules, and one or more stored algorithms.

In accordance with aspects of the present invention, the data transformation subsystem is further configured to collect and analyze data about user response to a nasal dilator.

In accordance with aspects of the present invention, the manufacturing subsystem uses additive manufacturing methods.

In accordance with aspects of the present invention, the manufacturing subsystem is configured to fabricate a mold for the personalized nasal dilator and use the mold to make a personalized nasal dilator.

In accordance with aspects of the present invention, the manufacturing subsystem is configured to modify an existing dilator to personalize the dilator for an individual user.

In accordance with embodiments of the present invention, a personalized nasal dilation system is provided. The personalized nasal dilation system includes one or more nasal inserts structurally adapted to align with a nasal passage surface anatomy of an individual user. Each insert includes one or more of: one or more varied configurations that fit nasal passage anatomical configurations of an individual user and a circumferential force profile configured to vary at different points of contact along a circumference of the insert providing one or more dilation force magnitudes disposed at different locations around the circumference. The one or more nasal inserts are shaped, sized, and configured based upon anatomical information of nasal passage surface features derived through an analysis of anatomical information for the individual user.

In accordance with aspects of the present invention, the one or more nasal inserts are formed of a pliant material.

In accordance with aspects of the present invention, the one or more nasal inserts are comprised of a biocompatible material.

In accordance with aspects of the present invention, each nasal insert includes a top opening, a base opening, and at least one support extending between the top opening and the base opening forming a passage extending between the top opening and the base opening. In some such aspects, the at least one support comprises a wall. In further such aspects, the wall comprises a mesh structure. In other aspects, the base opening is sized, dimensioned, and configured to engage a vestibular rim of a nose of a user.

In accordance with aspects of the present invention, the personalized nasal dilation system is configured as part of a CPAP pillow.

In accordance with embodiments of the present invention, a personal nasal dilator is provided. The personal nasal dilator results from acquiring data on an individual user's face and/or nasal structure, analyzing the data to evaluate anatomical and structural needs for the individual user, and manufacturing a nasal dilator device that is personalized for the individual user wherein the nasal dilator is configured, shaped, and dimensioned for the individual user based on the evaluated anatomical and structural needs of the individual user.

In accordance with embodiments of the present invention, a method for providing a personalized nasal dilator is provided. The method involves scanning an individual user's face and/or nasal structure, analyzing data for the individual user provided by the scanning to evaluate anatomical and structural needs for the individual user, and manufacturing a nasal dilator that is personalized for the individual user wherein the nasal dilator is configured, shaped, and dimensioned for the individual user based on the evaluated anatomical and structural needs of the individual user from a dilator profile. The scanning involves scanning an individual user's photogrammetry or 3D imaging and extracting landmarks from the scanning. The analyzing involves selecting a pro-forma dilator design based on the landmarks, modifying the pro-forma dilator design based on the landmarks to address anatomical and structural needs for the individual user, and generating a dilator profile.

In accordance with aspects of the present invention, analyzing the data further comprises collecting user profile data for the individual user. In other aspects, analyzing the data is performed using machine learning.

The invention, as further described in the claims section herein, is hereby incorporated by reference into this Summary.

### BRIEF DESCRIPTION OF THE FIGURES

These and other characteristics of the present invention will be more fully understood by reference to the following detailed description in conjunction with the attached drawings, in which:

FIG. 1 depicts the structure of the human nose;

FIG. 2 is a personalized nasal dilator in accordance with embodiments of the present invention;

FIG. 3 is a flow diagram illustrating a method of creating a personalized nasal dilator;

FIG. 4 is a system diagram illustrating the sub-systems utilized in creating a personalized nasal dilator;

FIG. 5 depicts 3D landmarks on a nasal structure;

FIG. 6 depicts a force profile of a nasal insert of a nasal dilator;

FIG. 7 depicts a configuration of a nasal dilator where the force profile is achieved by varying the structure and stiffness of various portions of the insert of a nasal dilator;

FIG. 8 depicts a front view of another embodiment of an insert for a nasal dilator system in accordance with embodiments of the present invention;

FIG. 9 depicts a side view of the insert of FIG. 8;

FIG. 10 depicts the coronal plane of the insert of FIG. 8;

FIG. 11 depicts the sagittal plane of the insert of FIG. 9;

FIG. 12 depicts the connector bridge used with the insert of FIG. 8 and FIG. 9;

FIG. 13 depicts the top view of the insert of FIG. 8 and FIG. 9;

FIG. 14 depicts the bottom view of the insert of FIG. 8 and FIG. 9;

FIG. 15 depicts example shapes for the top and base openings of the insert of FIG. 13 and FIG. 14;

FIG. 16 is a CPAP pillow configuration; and

FIG. 17 is an exemplary computing device that may be used in accordance with certain embodiments of the present invention.

### DETAILED DESCRIPTION

An illustrative embodiment of the present invention relates to a nasal dilator that is personalized by being uniquely configured, shaped, and dimensioned for a particular user. This personalized fit design is achieved by an analysis of data acquired that characterizes the nasal profile of the user, which can be derived from one or more of one or more scans or images of the user's face, exterior, and interior of nose and nostrils, and personal user profile information. The personalized fit nasal dilator is manufactured by a programmable manufacturing system, such as a digitally controlled process including additive manufacturing.

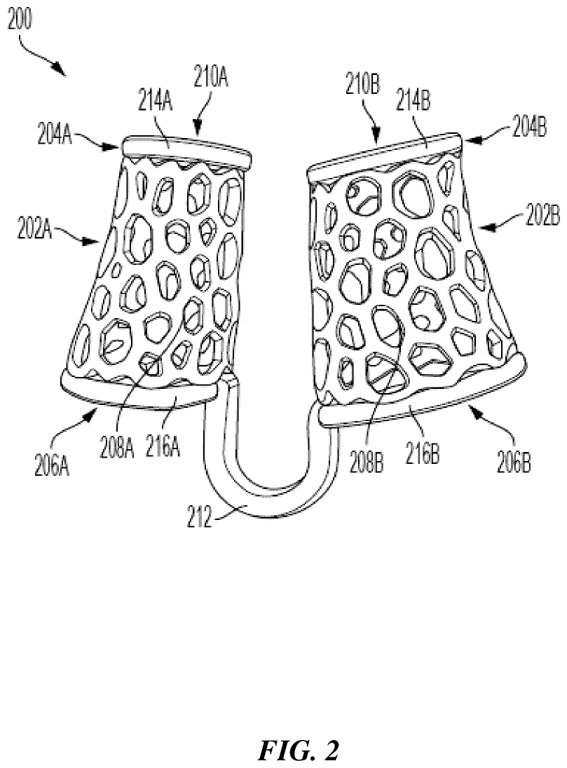

FIG. 2 through FIG. 17, wherein like parts are designated by like reference numerals throughout, illustrate an example embodiment or embodiments of a nasal dilation system and corresponding personalized nasal dilator, according to the present invention. Although the present invention will be described with reference to the example embodiment or embodiments illustrated in the figures, it should be understood that many alternative forms can embody the present invention. One of skill in the art will additionally appreciate different ways to alter the parameters of the embodiment(s) disclosed, such as the size, shape, or type of elements or materials, in a manner still in keeping with the spirit and scope of the present invention.

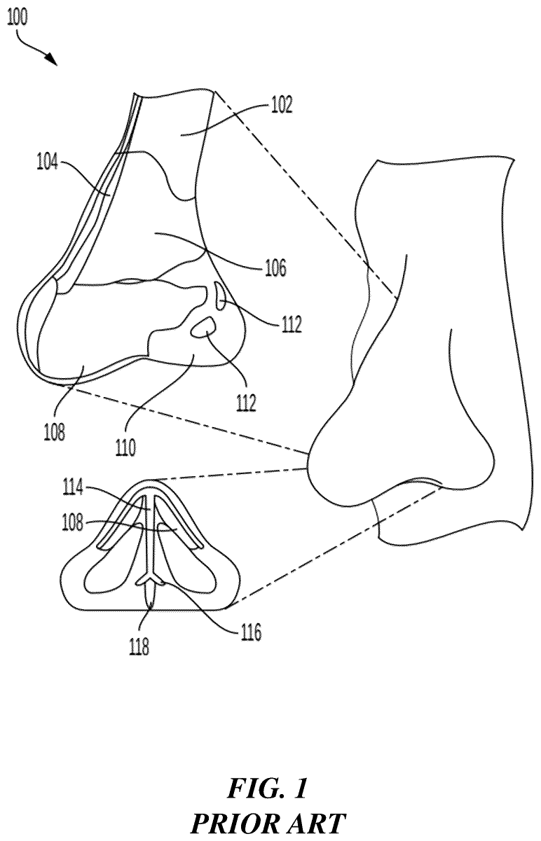

The human nose has a number of anatomical features that may affect the fit and operation of a nasal dilator. FIG. 1 shows the structure of a typical human nose 100. As can be seen here, the components that make up the structure of the nose include the nasal bone 102, septal cartilage 104, lateral nasal cartilage 106, greater alar cartilage (lateral crust) 108, alar fibrofatty tissue 110, minor alar cartilage 112, medial crus 114, footplate 116, and nasal septal cartilage 118. The human nose 100 has two valves: the internal and the external nasal valves. The internal nasal valve is level with the caudal border of the (upper) lateral nasal cartilage 106. This valve is a three-dimensional space bordered by the septum, the head of the lower turbinate, and the lateral cartilage and is not on a single plane. The internal nasal valve is the narrowest part of the entire airway. The external nasal valve includes all cartilaginous and soft tissue of the lateral nasal wall and that part of the septum, which is caudal to the internal nasal valve. A sub-component of the external nasal valve can be considered to be the vestibular valve at the level of the vestibular rim. While some might describe the vestibular valve to be a discreet entity, we include it in our definition of the external nasal valve.

It is well known to those skilled in the art that nose morphologies, including the two nasal valves, vary from person to person and that some traits can be common across different cohorts, which can be defined along different sets such as age, gender, racial or genetic lines, or simply morphological such as “bulbous,” “hook,” “pointed,” etc.

In addition, alterations in nose morphologies can be specific to an individual based on genetics, birth defects, diseases, surgical procedures, physical trauma, or the like. Furthermore, the morphologies or anatomical structure of individual nostrils of the individual can vary for any of the above reasons.

The approach of the present invention results in a nasal dilation system comprising one or more nasal inserts structurally adapted to align with a nasal passage surface anatomy for each nostril of a specific individual. Each insert comprises one or more varied configurations that fit nasal passage anatomical configurations specific to the particular user and/or a circumferential radial force profile configured to vary at different points of contact along a circumference of the insert, providing one or more dilation force magnitudes disposed at different locations around the circumference at different depths of insertion. The one or more nasal inserts are shaped, sized, and configured based on anatomical information of nasal passage surface features derived through an analysis of anatomical information obtained from scans of the individual.

The resulting personalized nasal dilator of the present invention provides improvement of breathing by nasal dilation, that is, reducing resistance to airflow caused by constriction, achieved both by widening or opening and by holding open the nasal valve area of the nose. Constriction of the nasal valve can be continuous, e.g., in someone with a collapsed or narrowed nasal valve (common after trauma and in old age, but also congenitally) or rhythmic, caused by the soft tissue of the nasal valve being “sucked in” by the venturi effect of breathing in. Nasal valve constriction can also be temporarily increased by swelling in the nasal passages due to various causes, including injury, inflammation, and infection. Of the many manifestations of constricted nasal valves, the two most (but not only) likely impacted by the invention are i) snoring and/or sleep disturbance, including sleep apnea, and ii) athletic performance.

Nasal valve constriction can lead a person to learn to more frequently mouth breathe instead of nose breathing.

Thus, the target users for the inventive device include:
People who, either consistently or intermittently, snore or suffer other breathing-related sleep disturbances, for whom the device may reduce snoring and breathing-related sleep disturbances;
People with sleep apnea: while the device may not be sufficient to stop sleep apnea, it may help to reduce the severity and frequency of occurrences; the device is not a substitute for other sleep apnea treatments, such as a Continuous Positive Airway Pressure machine (CPAP);
Athletes of all levels of ability who wish to enhance their cardiovascular performance by improved breathing enabled by using the device in training or in competition;
Athletes of all levels of ability who wish to learn to nose breathe more often during exertion, using the device as a training aid;
People who wish to learn to nose breathe more often in any situation, using the device as a training aid;
People who already use internal nasal dilators but who suffer from discomfort or skin/tissue health issues as a result of such use;
People who use external nasal dilators who may find that this device works better for them without introducing the disadvantages of other internal nasal dilators;
People with temporary nasal constriction caused by injury, inflammation, infection, or other reasons who wish to improve breathing in any circumstance, awake or asleep; and/or
In the device CPAP pillow form, people who use CPAP machines, making the pillow more comfortable and increasing airflow for a given pressure setting on the machine.

In some instances, the personalized nasal dilator of the present invention may also provide relief from nasal congestion.

In the embodiment of FIG. 2, each insert 202A, 202B includes a top opening 204A, 204B and a base opening 206A, 206B connected by at least one support 208A, 208B extending between the top opening 204A, 204B and base opening 206A, 206B forming a passage 210A, 210B or cannula extending between the top 204A, 204B and base 206A, 206B openings.

In certain embodiments, the top openings 204A and 204B are defined by upper rings 214A and 214B, while the base openings are defined by base rings 216A and 216B. The top opening 204A, 204B and base opening 206A, 206B may have any number of shapes and sizes, including wherein the top opening 204A, 204B and base opening 206A, 206B may even have different sizes or shapes. In even further embodiments, the top opening 204A and/or base opening 206A of one insert 202A may have different sizes or shapes from the top opening 204B and/or base opening 206B of the other insert 202B.

In certain embodiments, the at least one support 208A, 208B may comprise a wall. In some such embodiments, the at least one support 208A, 208B may comprise a mesh or cage structure.

In certain embodiments, the nasal dilation system 200 is formed of one or more of metal, plastic, polymer, and rubber. In certain embodiments, the nasal dilator system 200 is formed of a pliant material. In certain embodiments, the one or more nasal inserts 202A, 202B are comprised of a biocompatible material, such as, for example, silicone, polyurethane, polyethylene terephthalate (PET), and polylactic acid (PLA). Those of ordinary skill in the art will appreciate that other biocompatible materials can be utilized in accordance with the requirements and teachings of the present disclosure.

It is the combination of the top opening 204A, 204B, base opening 206A, 206B, and at least one support 208A, 208B that provides the personalized nasal dilator. That is, the personalized size, shape, and configuration of each of the top opening 204A, 204B, base opening 206A, 206B, and at least one support 208A, 208B combine to make the personalized nasal dilator. These can include curves, indents, bulges, and other variations in the size, shape, and configurations of the top opening, base opening, and at least one support.

In FIG. 2, the top opening 204A, 204B and the base opening 206A, 206B have different sizes and shapes. The support 208A, 208B is a mesh wall extending from the top opening 204A, 204B to the base opening 206A, 206B. As can be seen here, the mesh wall also has varying shapes and configurations. Here, the inserts 202A, 202B are further connected by a connector bridge 212.

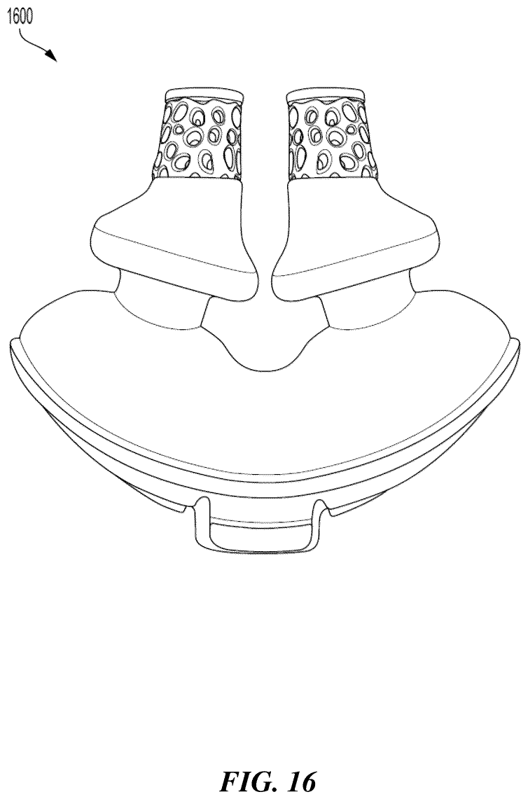

The invention is equally applicable to use in a CPAP “pillow,” where the same design process used for the nasal dilator profiling is used to create a more comfortable pillow insert, thus effecting a higher nasal patency and increased comfort. An example of this can be seen in FIG. 16.

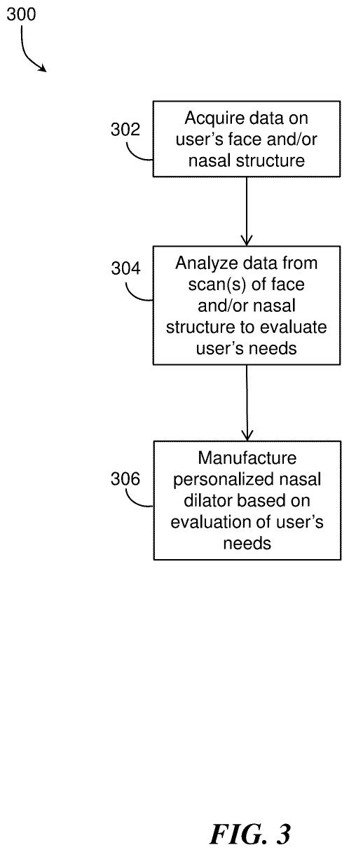

The approach of the present invention is set forth at a high level in flowchart 300 of FIG. 3. This involves acquiring data characterizing the individual user's face and/or nasal structure (Step 302), analyzing the data for the individual user to evaluate the individual user's anatomical and structural needs (Step 304), and manufacturing a nasal dilator that is personalized for the individual user wherein the nasal dilator is uniquely configured, shaped, and dimensioned for the individual user based on the evaluated anatomical and structural needs of the individual user (Step 306).

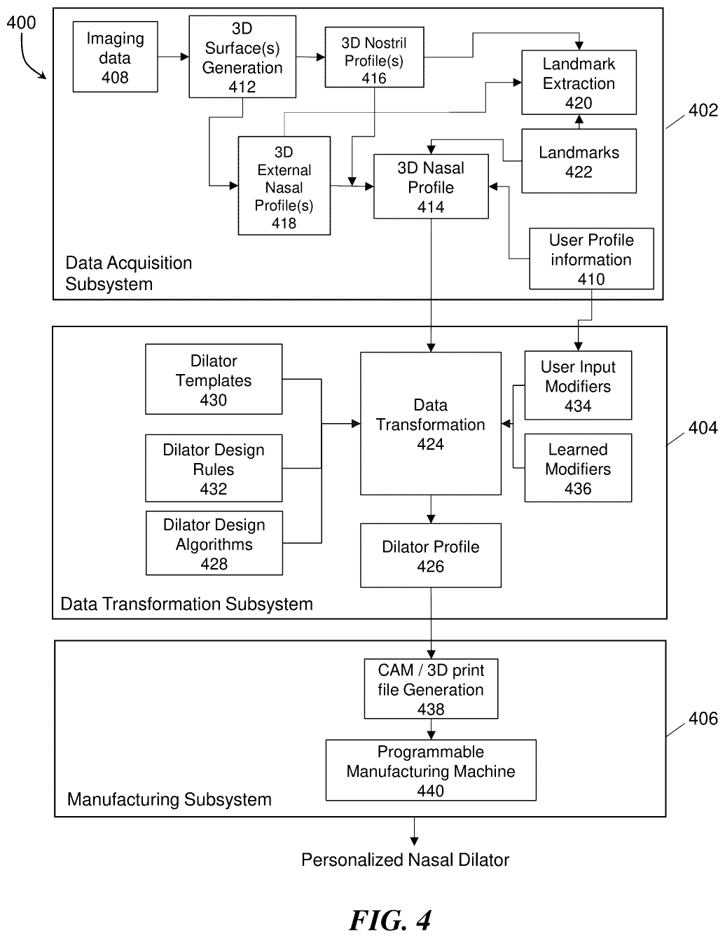

The components or sub-systems used to perform the approach set forth in flowchart 300 of FIG. 3 can similarly be grouped into three groups based on functionality, as shown in system 400 of FIG. 4. These include data acquisition subsystems 402, data transformation subsystems 404, and manufacturing subsystems 406.

### Data Acquisition Subsystem(s)

The Data Acquisition Subsystem 402 is configured to receive information (including imaging data 408 and user profile information 410) about a user's facial and nasal structures and generate a 3D Nasal Profile (414) of a user's nose and nostrils. The 3D Nasal Profile 414 includes one or more 3D Nostril Profiles 416 comprising 3D profile of the internal of the individual user's nostrils up to, and optionally including, the internal nasal valve, one or more 3D External Nasal Profiles 418, one or more sets of Landmarks 422 extracted (420) from one or more of the one or more 3D Nostril Profiles 416 and the 3D External Nasal Profiles 418 and User Profile Information.

The imaging data 408 about a user's facial and nasal structures can be photographs or scans obtained using a camera or camera-equipped personal electronic device or imaging data acquired from a third party such as an imaging center. The personal user profile information 410 can include influential factors such as age, sex, weight, height, smoking/alcohol use, race or ethnicity, snoring type, nose shape, desired outcomes, etc. In some such embodiments, the personal user profile information 410 is obtained from the user in response to questions. In another embodiment, the user profile information includes nasal morphologies and sizing derived by the user via comparisons and measurements made with templates or drawings. In still other embodiments, the personal user profile information can further include information about the performance or user experience of one or more nasal dilators used by the user.

In one embodiment, 3D Surface Generation 412 is provided by video or photo photogrammetry (of one or more of the user's face, exterior of nose and nostrils, and interior of nose and nostrils) to build one or more of one or more of 3D Nostril Profiles 416 and a 3D External Nasal Profiles 418. Photogrammetry is a technique that can be built upon the images or video from any digital camera system, such as that on the user's personal device, e.g., a mobile phone.

An alternative to photogrammetry is to deploy any of the other available 3D imaging techniques available on some devices like Apple's recent devices (e.g., their TrueDepth™ cameras used for face ID™) and some Samsung devices, such as so-called LiDAR and depth or time of flight (TOF) cameras using a variety of known 3D imaging techniques such as infrared (IR) time-of-flight, structured (IR) light patterns, and multiple/stereo cameras.

In another example embodiment, alternative energy imaging systems can be used, including X-ray, computed tomography (CT) systems, ultrasound imaging, and other known imaging modalities. Cone beam CT is particularly useful because it is readily available at some dental practices and at a modest cost. The advantage of such systems over visible photogrammetry is that imaging of deeper and occluded structures within the nostril is feasible, whereas photogrammetry may require more attempts to record sufficient data to build a 3D Nostril Profile 416.

Another known technique that can be used is acoustic rhinometry, in which a partial 3D profile 416 of the nostril is built using acoustic time domain reflectometry techniques. Clinical acoustic rhinometers are available at some clinics, and a home-use unit is feasible. With a small and low-cost ancillary tool, acoustic rhinometry can be performed on a personal device such as a mobile phone. Acoustic rhinometry can be used as a stand-alone imaging technique or can be used to augment a 3D Nostril Profile 416 derived from another technique, such as those described herein. This combination approach is particularly useful with visible photogrammetry, where missing 3D profile data from either technique alone can be supplemented by data from the complementary technique, thus rendering a more complete 3D Nostril Profile 416.

The 3D Nostril Profile 416 may be derived directly from one or more of the described techniques, where a 3D Nostril Profile 416 of the interior of the nostril is generated directly from the scanned images. In other embodiments, the 3D Nostril Profile 416 is fabricated from a combination of scans of the exterior of the nose and/or face of the user, optionally augmented with interior nostril scans and 3D profiles. This embodiment is particularly useful where interior 3D scanning is unable to build a 3D Nostril Profile 416 of sufficient quality from the 3D profile data for any of several reasons, including narrow nasal cavities, deviated septums, poor lighting conditions, user error, etc.

In data-rich, information-sparse, imaging techniques such as photogrammetry or combined photogrammetry/acoustic rhinometry, adaptive algorithms such as machine learning types can be trained to create higher quality 3D Nostril Profiles 416 than achievable by fixed image processing and data fusion algorithms. Such a machine learning approach may be trained on ground truth data derived from alternative energy imaging modalities such as x-ray CT described above.

Photogrammetry calibration is achieved using a combination of: camera calibration, camera calibration databases, camera auto-calibration techniques known to those skilled in the art, optionally augmented by additional calibration provided by in-view calibration marks, such as printed fiducials or scales, or common objects of defined size such as credit cards, coin, etc., any of which can be held in place during image acquisition by the user or by use of a suitable removable adhesive. In a further embodiment, data from additional sensors on a user's mobile device, such as inertial, GPS, acoustic, and capacitive, may be used to calibrate the photogrammetry-derived 3D Nostril Profiles 416 and 3D External Nasal Profiles 418.

In another embodiment, in combination with any of the imaging techniques used to build a 3D Nostril Profile 416 and a 3D External Nasal Profile 418, the user acquires supplemental “posed” images and/or 3D profile scans that are used to augment the data transformation by providing additional information on the user's unique physiology. One such supplemental posed image or scan is taken while the user wrinkles their nose (so-called “bunny nose”). Another such supplemental posed image or scan is taken while the user flares their nostrils. A third such posed supplemental image or scan is taken while the user forcefully inhales through their nose. A fourth such supplemental posed image or scan is taken while the user holds one nostril, then the other, either closed or stretched open by use of a finger or other force provider. There may be other supplementary posed images of scans, and any combination of none, one or more supplemental images may be used to augment the baseline scan.

The 3D Nostril Profile 416 and 3D External Nasal Profile 418 can be represented in any of a number of known techniques, including point clouds, polygonal or other geometric meshes, wireframe, b-splines, vector graphics, OBJ, STL, STEP, IGES, CAD, and others. Those skilled in the art will recognize that many possible representations are possible and that the method taught herein is independent of the choice.

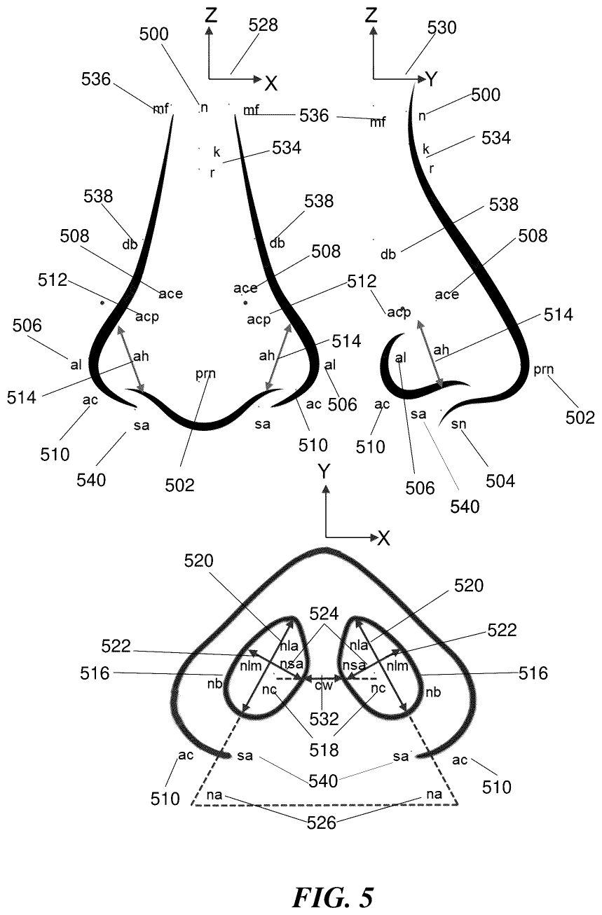

In certain embodiments, a set of Landmarks depicted in FIG. 5, comprising 3D coordinates of specific anatomical features, is created by well-known image processing techniques and/or feature extraction techniques acting on either raw 2D or 3D images or any of the known representations of 3D profiles outlined above. Land marks may include one or more of: Nasion (n) 500, Pronasale (prn) 502, Subnasale (sn) 504, Right & Left Alar (al) 506, Right & Left Supra end alar crease (ace) 508, Right & Left Alar Crest (ac) 510, Right & Left Alar crease plot (acp) 512, Right & Left Alar Lobule height (ah) 514, Right & Left Nostril Circumference (nb) 516, Right & Left Nostril Centroid (nc) 518, Right & Left Nostril long axis length (nla) 520, Right & Left Nostril long axis mid-point (nlm) 522, Right & Left Nostril short axis length (nsa) 524, Right & Left Nasal Angle (na) 526, Right & Left Nostril plane angle @ X axis (npx) 528, Right & Left Nostril plane angle @ Y Axis (npy) 530, Columellar width (cw) 532, Kyphion or—better—Rhinion (k,r) 534, Right & Left Maxillofrontale (mf) 536, Right & Left Dorsal base (db) 538, Right & Left Sub Alar (sa) 540, Right & Left Int valve depth, Right & Left Int valve length, Right & Left Int valve width, Right & Left Int valve angle. The anatomical and/or image processing algorithmic definition of each of these Landmarks is well defined in the literature. In some instances, slight variations may be found in the definitions in the literature and the precise definition used is immaterial to the operation of the method, so long as definitions are applied consistently. Those skilled in the art will recognize that there are other landmarks that could be used in addition or in substitution for one or more of these Landmarks

### Data Transformation Subsystem(s)

Data transformation subsystems 404 include one or more data transformations 424 that are configured to process the scan or imaging data and extracted landmarks and/or user profile information to evaluate the user's nasal characteristics for the creation of a Nasal Dilator design. The data transformations 424 of the data transformation subsystems 404 are used to create an output Dilator Profile 426 based upon the inputs of one or more of the 3D Nasal Profile 414 and any supplemental posed images or scans collected by the user, and User Input Modifiers 434 derived from the user profile information 410, which includes influential factors such as age, sex, weight, height, smoking/alcohol use, race or ethnicity, snoring type, nose shape, desired outcomes, etc.

In accordance with one example embodiment, the Dilator Profile 426 is created as a geometric transformation from the one or more 3D Nostril profiles 416 of the one or more 3D Nasal Profiles 414, for example, a scaled transformation, or one scaled differently in different zones of the profile, such as the upper 214 and base 216 dilator rings.

In another embodiment, the data transformation takes, instead of the 3D Nasal Profiles 410, the various raw 3D scans (of one or more scans of the user's face, exterior and interior of nose and nostrils) along with any supplemental posed images or scans collected by the user, plus user profile information and acts directly on these to generate the output Dilator Profile 426 without generating a discrete data entity of a 3D Nasal Profile 410.

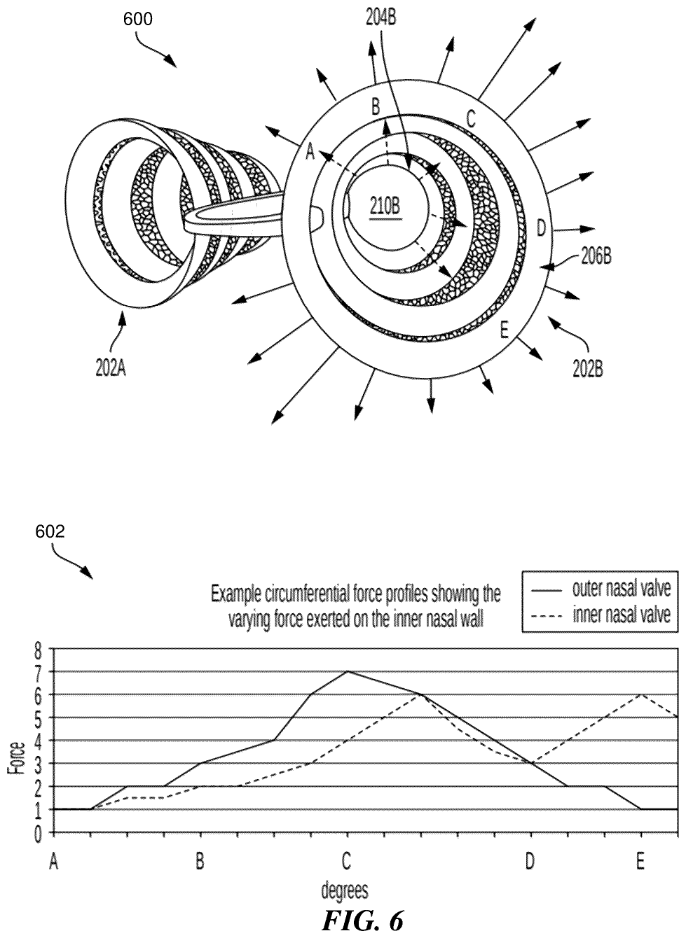

In certain embodiments, a Dilator Profile 426 is created where the dilation force is designed to differ at various points on the circumference of one or both of the rings 214, 216, thus creating one or two circumferential radial force profiles. The circumferential radial force profiles can be designed to optimally trade off comfort and nasal patency (referring to an assessment of the openness or lack of obstruction of a nasal passage) by applying sufficient force at each point around the circumference to achieve good nasal patency without uncomfortable or excess force. The circumferential radial force profile can be designed to accommodate the variations in the underlying anatomical structure and soft tissue tone around the circumference, as well as the impact of dilation amount on nasal patency, which also varies around the circumference. The design or selection of optimum circumferential radial force profiles can be part of the data transformation subsystem 404. The varying dilation force around the circumference may be achieved by one or more of a number of approaches, including geometric changes such as thickness, material type, material property modification, and mechanical structures such as lamination. An example of this can be seen in FIG. 6. In force depiction 600, the dilation force, indicated by the arrows, varies along the circumference as well as along the length of the insert 202B as indicated by the respective length of the arrow indicating the magnitude of the dilation force. Force plot 602 shows the magnitude of the dilation force around the circumference of the insert 202B.

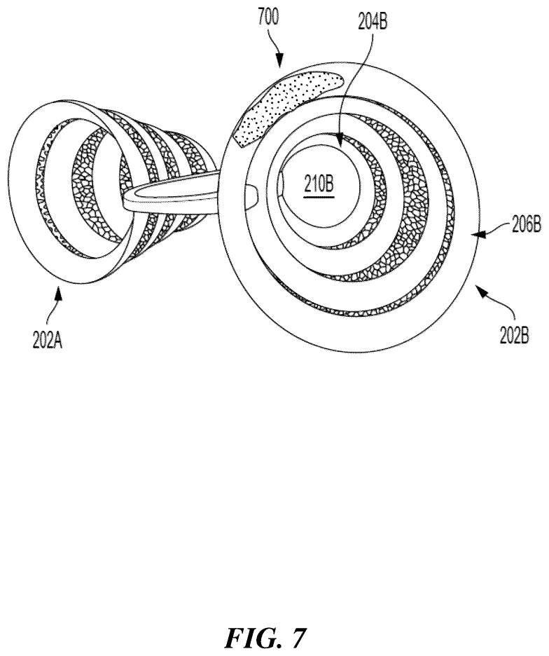

In one example, as seen in FIG. 7, the radial force profile of the structure of the base opening 206B varies around its circumference to exert a relatively larger force against the lateral processes of the greater alar/lateral crus cartilage 108, thus preventing inward collapse of the external valve by inward movement of the lateral crus cartilage 108; while also exerting a relatively lesser force against the minor alar cartilage 112 and the upper portions of the alar fibrofatty tissue 110, thus avoiding discomfort there. In this embodiment, the radial force profile is obtained by the use of a stiffer material 700 in the anterior portion of the structure of the base opening 206B, combined with a thinner amount of material in the lateral and medial sections of the opening structure. Similarly, the structure of the top opening 204B may be profiled, so as to exert relatively more force against the lower alar fibrofatty tissue 110 and relatively less force against the septal cartilage 104. Those skilled in the art will recognize that other circumferential radial force profiles would be appropriate for different users' nasal anatomical shapes.

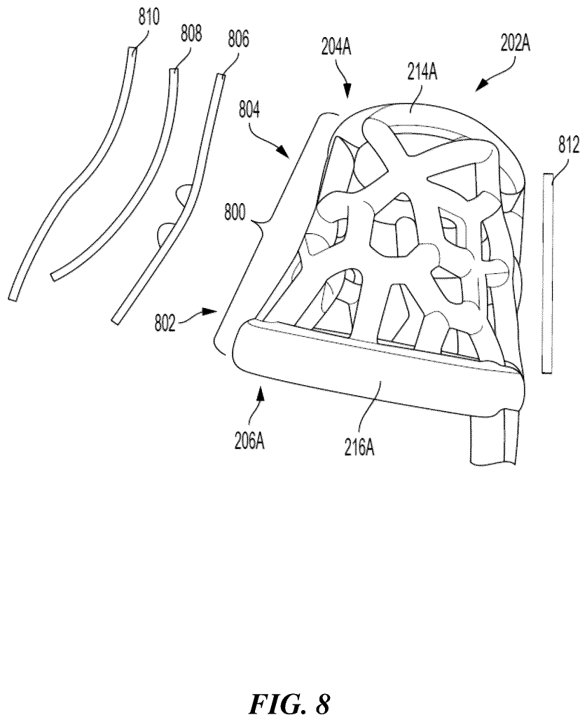

FIGS. 8-14 depict some such embodiments of the nasal dilation system 200 of the present invention in which the data transformation subsystem 404 includes stored algorithms 428, stored templates 430, or stored rules 432.

FIG. 8 depicts a front view of an insert 202A. In this embodiment, the lateral longitudinal profile 800 is designed to provide a proportionate opening force on the external nasal valve, i.e., the greater alar cartilaginous 108 structure and lesser/minor cartilaginous 112 structure, which in turn will exert a force on the inner nasal valve, i.e. lateral nasal cartilage structure 106 and septal cartilaginous 118 structure, rather than exerting most of the force on these superior cartilaginous structures directly by the dilator insert 202A. The insert profile 800 in the coronal plane is such that the tapering angle of the insert 202A is greater in the lower/caudal part 802 toward the base opening 206A than the higher/superior part 804 toward the top opening 204A. This can be achieved via a freeform design or a number of mathematical shapes, including lines interconnected by one or more vertices 806, an arc 808, a hyperbola, an “S” curve, a partial sine or hyperbolic sine shape 810, and others known to those skilled in the art. The medial profile 812 is designed to conform to the relatively straighter sagittal plane lateral aspect of the septal cartilage 104, 118 surface of the nostril 100 relative to the internal surface of the ala 108, 112.

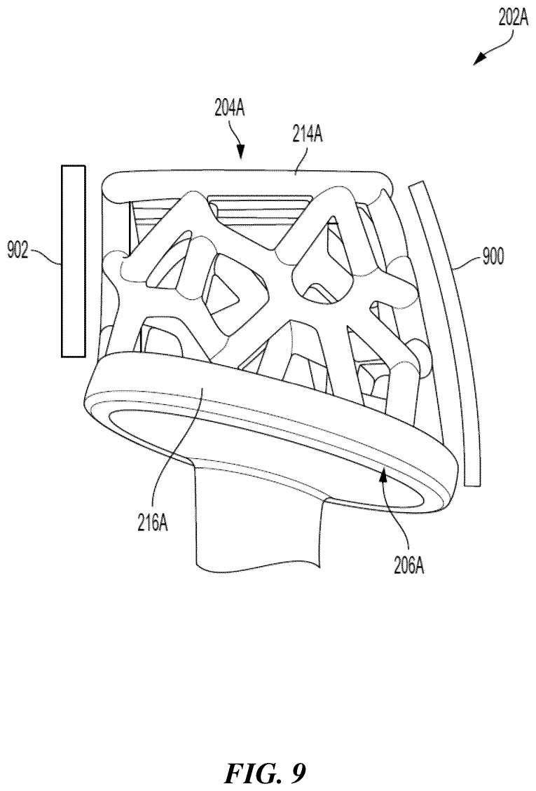

FIG. 9 depicts a side view of the insert 202A. Here, the graduated taper profile 800 is designed to conform to the anterior curve of the nostril interior due to the shape of the nasal ridge formed by the profile of the septal cartilage 104 in the sagittal plane relative to the posterior surface of the insert 202 designed to engage with the posterior surface of the nostril. The profile 900 is also designed to be different from the lateral longitudinal profile 800 so as to conform to the medial surface of the septal cartilage 104, 118 of the nostril 100 relative to the internal surface of the ala 108, 112. This can be achieved via freeform design or via a number of mathematical shapes, including those depicted in FIG. 8, including lines interconnected by one or more vertices 806, an arc 808, a hyperbola, an “S” curve, a partial sine or hyperbolic sine shape 810, and others known to those skilled in the art.

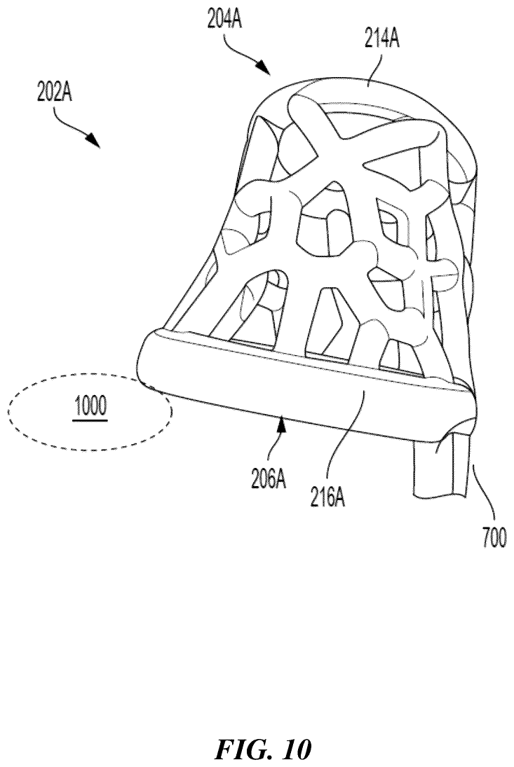

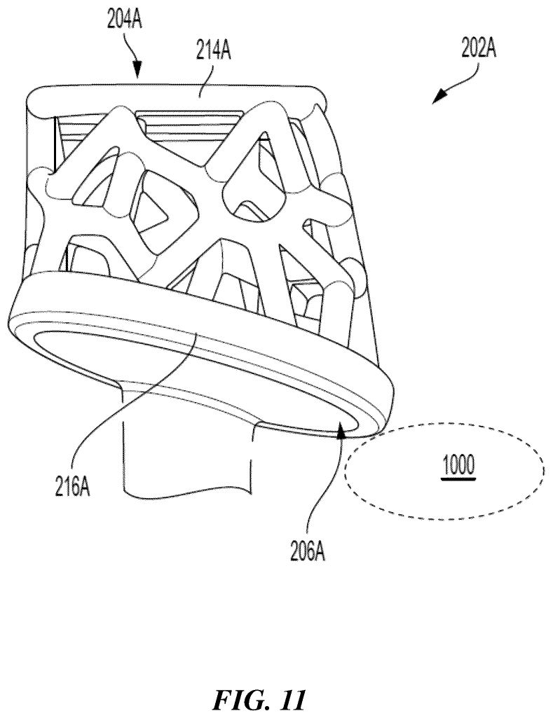

The insert 202A is designed to fit snugly inside a nostril with the base ring 216A defining the base opening 206A of the insert 202A positioned fully superior to the vestibular rim 812 of the nostril opening immersed, such that the vestibular rim 1000 partially engages the lower/caudal surface of the base ring 216A of the base opening 206A of the insert 202A. The angles of the base ring 216A of the base opening 206A of the insert 202A to the medial septum edge of the insert 202A in both the coronal and sagittal planes are such as to engage the base ring 216A of the base opening 206A uniformly within the vestibular rim 1000. Examples of this can be seen in FIG. 10 and FIG. 11. The angles of the base ring 216A of the base opening 206A are derived through an analysis of anatomical information obtained from scans of the individual.

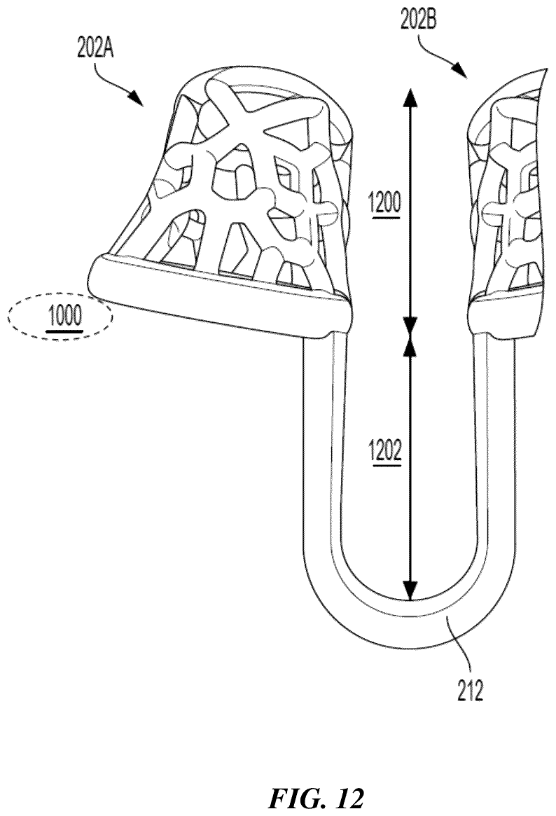

FIG. 12 depicts the connector bridge 212 used with this embodiment of the nasal dilation system 200. In certain embodiments, the length of the connecting bridge 1202 is proportionately greater than the length 1200 of the insert 202A, 202B to ensure that the inserts 202A, 202B are fully inserted into the nostril superior to the vestibular rim 702 such that the vestibular rim 1000 partially engages the lower/caudal surface of the base ring 216A, 216B of the base opening 206A, 206B of the inserts 202A, 202B with no interference between the bridge 212 and the nasal columella and/or medial crus 114 of the nose 100. The bridge length 1202 is derived through an analysis of anatomical information obtained from scans of the individual.

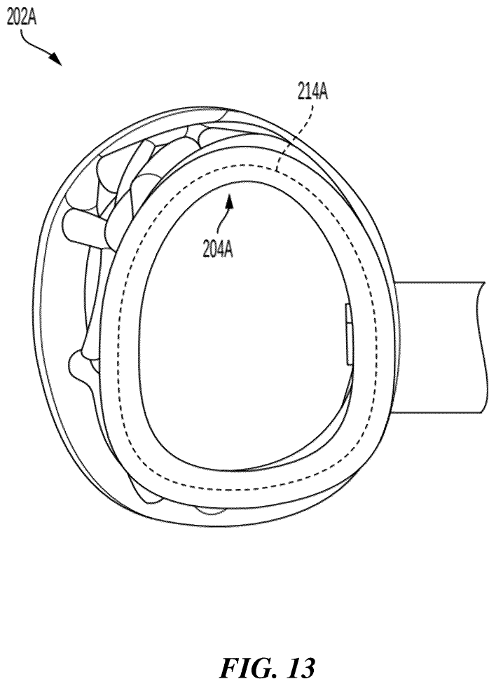

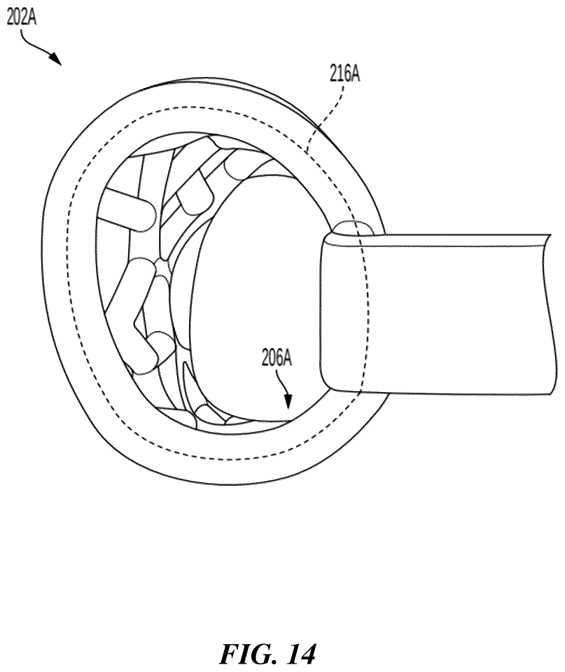

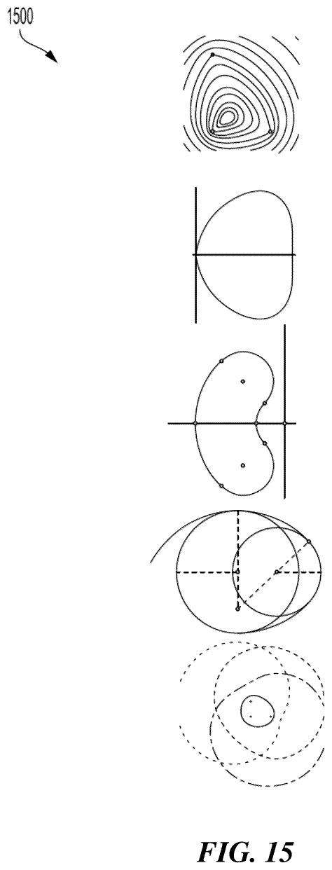

FIG. 13 depicts a top view of the insert 202A, while FIG. 14 depicts a bottom view of the insert 202A. In certain embodiments, the shape of the top ring 214A at the top opening 204A and base ring 216A at the base opening 206A are non-uniform, not approximately circular, or irregular in shape in order to better conform to the actual shape of a user's nostril as derived through an analysis of anatomical information obtained from scans of the individual. In some embodiments, one or both of the top ring 214A and base ring 216A have a generally ellipse shape. In other embodiments, one or both one or both of the top ring 214A and base ring 216A are formed in the shape of an oblong. In still other embodiments, one or both of the top ring 214A and base ring 216A are formed in the shape of an ovoid or egg. In further embodiments, one or both of the top ring 214A and base ring 216A have the shape of a 3-ellipse. In still further embodiments, one or both of the top ring 214A and base ring 216A have the shape of a n-ellipse, where n>3. In yet further embodiments, one or both of the top ring 214A and base ring 216A have the shape of a nephroid, fabiform, or bean curve. In alternate embodiments, one or both of the top ring 214A and base ring 216A are formed in a totally irregular, broadly convex shape or a totally irregular, broadly convex shape containing one or more concavities and other shapes known to those skilled in the art. Examples of some possible mathematical shapes 1500 can be seen in FIG. 15. The selection of the shape form and parametrization of the top ring and base ring are derived through an analysis of anatomical information obtained from scans of the individual.

In certain embodiments, the data transformation 424 additionally takes as input copies of one or more pro-forma dilator designs and modifies these according to data derived from the 3D Nasal Profile 414 or the raw scans or the User Profile Information 410 from which to produce a Dilator Profile 426. These pro-forma dilator designs are derived from combinations of one or more of one or more stored templates 430, one or more stored rules 432, and one or more stored algorithms 428, the selection of which is determined algorithmically from the 3D Nasal Profile 414 or the raw scans or the User Profile Information 410.

In one such embodiment, a Dilator Profile 426 is created from a pro-forma design template such as those depicted in FIGS. 5-13 which is modified according to the relationship between the Landmarks 422 in the user's 3D Nasal Profile 414 and the corresponding landmarks associated with that pro-forma design. In one embodiment, the modification is a simple linear scaling by one or more of the Landmarks 422, or distances or angles between them. By way of example, if the Nasion (n) 500 to Pronasale (prn) 502 inter-landmark distance in the 3D Nasal Profile 410 is 10% greater than the nasion 500 to pronasale 502 inter-landmark distance associated with the template, then the length of the dilator as illustrated by the profiles 800, 812 and 900, 902 might be increased by 10%, or some other scale factor as determined by the proforma rules. Similarly, the base ring 216A, in whatever mathematical shape or totally irregular shape it may be, may be scaled according to the nostril long axis length (nla) 520, nostril long axis mid-point (nlm) 522, nostril short axis length (nsa) 524, nasal angle (na) 526 and nostril centroid (nc) 518 contained in the user's 3D Nasal Profile 414, relative to those features and dimensions associated with the template 430. Similarly other lengths, sizes, shapes and profiles of the pro-forma template 430 design may be scaled according to other combinations of Landmarks 422 and inter-landmark distances in the user's 3D Nasal Profile 414 relative to the corresponding landmarks and inter-landmark distances associated with the template.

In some such embodiments, a template 430 of a specific size, shape or profile might be selected from a set of templates 430, according to the Landmarks 422 and inter-landmark distances in the User's 3D Nasal Profile 414. These such selected templates 430 might further be scaled as above.

Those skilled in the art will recognize that other, non-linear relationships might be beneficial to the scaling of a pro-forma template 430 according to the relationship between the Landmarks 422 in the 3D Nasal Profile 410 and landmarks associated with the pro-forma template 430.

In a further embodiment, the pro-forma dilator is represented by design rules 432 and/or algorithms 428 acting upon landmarks which are then applied or calculated using the Landmarks 422 in the user's 3D Nasal Profile 414 to create a Dilator Profile 426.

In further embodiments, the data translation 424 modifies a copy of the 3D nasal profile 414 or raw scans, using combinations of one or more of one or more of one or more stored templates 430, one or more stored rules 432, and one or more stored algorithms 428, the selection of which is determined algorithmically from the 3D Nasal Profile 414 or the raw scans.

In further embodiments, the Data Transformation 424 is modified according to one or more User Input Modifiers 434 based on the user profile information 410, which can include influential factors such as age, sex, weight, height, smoking/alcohol use, race or ethnicity, snoring type, nose shape, desired outcomes, etc. Such modifications can be applied to any aspect of the transformation, including scaling factors, templates, design rules, and design algorithms.

One skilled in the art would understand that any of the above examples could be combined or otherwise used in a reciprocal method.

The relationship between the individual's anatomical nasal geometries represented by the 3D Nasal Profile 414 or raw scans and the optimum personalized fit profile and circumferential force profiles represented by the Dilator Profile is a complex one derived by an analysis of anatomical data and/or from empirical data derived from experimentation and/or collection of Nasal Dilator efficacy data from users of their own personalized dilator devices, and optionally by use of machine learning techniques, optionally adapting to new data acquired by analysis of the user and by their response to the use of the nasal dilation system 200 or to new data acquired by analysis of other users and by their responses to the use of the nasal dilation system 200. Any number of machine learning techniques known to those skilled in the art may be used, including either supervised, unsupervised, or reinforcement learning using methods such as artificial neural networks, convolutional neural networks, Bayesian models and classifiers, decision trees, support vector machines, random forests, regression, K means regression. The use of machine learning algorithms to determine the Data Transformation 424 may be self contained or may be applied via means of Learned Modifiers 436 to any and all of the data Transformations 424 described herein. This adaptable relationship forms one of the aspects of the current invention.

The Data Transformation 424 itself is provided by a processing machine having access to the translation algorithms and user input data. This processing machine can be a machine local to the user such as the device used to acquire scans or a personal computer or can be a central or distributed machine connected to the user's input scanning device by appropriate communication means including internet, Wi-Fi, cell phone, or other means. The processing machine may entail multiple steps that may not all be provided by the same processor and may engage third-party processing machines. Examples of such steps include photogrammetry processing to create 3D profiles, creation of manufacturing machine control files, etc.

### Manufacturing Subsystem(s)

The manufacturing subsystems 406 are configured to fabricate customized files for the nasal dilation apparatus 200 based on the evaluated nasal characteristics. 3D Print Files, Computer Aided Manufacturing (CAM) files or similar can be generated 438 from the Dilator Profile 426, which can be used to control a programmable manufacturing machine 440 such as a digitally controlled process one of which is an additive manufacturing machine, to fabricate the personalized internal nasal dilator.

Any of a number of techniques known to those skilled in the art may be used to fabricate the device, including photopolymerization, or other polymerization, powder bed, and filament extrusion additive manufacturing systems.

The materials may be selected from a range of medical-grade polymers, including silicone rubbers or elastomers. There may be benefits in manufacturing a dilator with more than one material, which can be achieved with certain additive manufacturing systems.

In one example embodiment, a programmable manufacturing system can be used to fabricate a mold from some other material (polymer, ceramic, metal, etc.) that is then used to mold the personalized internal nasal dilator in a non-programmable process such as injection molding, blow molding, casting. This embodiment can be especially useful where multiple repeat orders for replacement devices is anticipated, making manufacturing less costly in these cases.

In one example embodiment, personalized internal nasal dilators can be created by a modification to one of a set of standard “blank devices, e.g. via stretching, bending, compressing, or otherwise distorting and then setting the new shape, for example with a thermo, ionization, or photonic based method to freeze or re-polymerize the material in its new shape.

In certain embodiments, the nasal dilator is formed of one or more of metal, plastic, polymer, and rubber. In certain embodiments, the nasal dilator is formed of a pliant material. In some certain embodiments, the one or more nasal inserts are comprised of a biocompatible material, such as for example, silicone, polyurethane, polyethylene terephthalate (PET), and polylactic acid (PLA). Those of ordinary skill in the art will appreciate that other biocompatible materials can be utilized in accordance with the requirements and teachings of the present disclosure. A nasal dilator may be manufactured with more than one material either in a single step, or in multiple steps in the process.

The invention is equally applicable to use in a CPAP “pillow”, where the same design process used for the nasal dilator profiling is used to create a more comfortable pillow insert effecting a higher nasal patency. An example of this can be seen in a dilator 1600 FIG. 16.

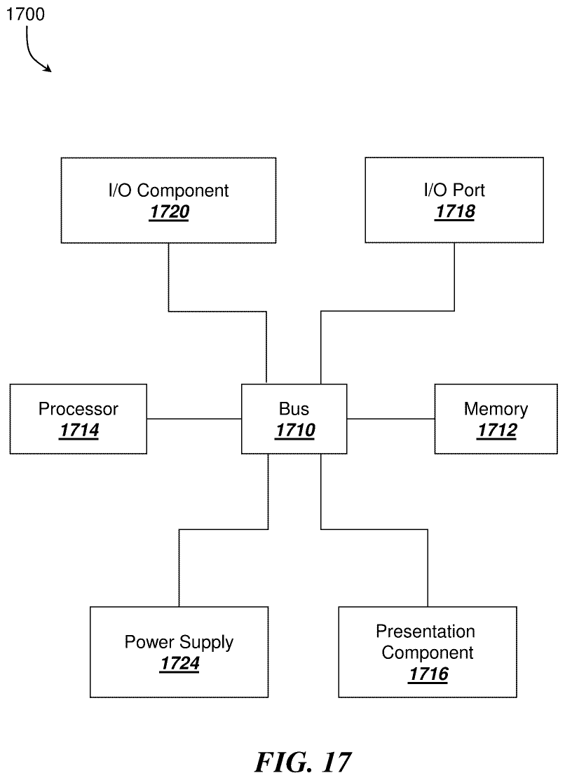

Any suitable computing device can be used to implement the system 400 or subsystems including the scanning subsystems 402, data transformation subsystems 404, and the manufacturing subsystems 406. One illustrative example of such a computing device 1700 is depicted in FIG. 17. The computing device 1700 is merely an illustrative example of a suitable computing environment and in no way limits the scope of the present invention. A “computing device,” as represented by FIG. 17, can include a “workstation,” a “server,” a “laptop,” a “desktop,” a “hand-held device,” a “mobile device,” a “tablet computer,” or other computing devices, as would be understood by those of skill in the art. Given that the computing device 1700 is depicted for illustrative purposes, embodiments of the present invention may utilize any number of computing devices 1700 in any number of different ways to implement a single embodiment of the present invention. Accordingly, embodiments of the present invention are not limited to a single computing device 1700, as would be appreciated by one with skill in the art, nor are they limited to a single type of implementation or configuration of the example computing device 1700.

The computing device 1700 can include a bus 1710 that can be coupled to one or more of the following illustrative components, directly or indirectly: a memory 1712, one or more processors 1714, one or more presentation components 1716, input/output ports 1718, input/output components 1720, and a power supply 1724. One of skill in the art will appreciate that the bus 1710 can include one or more busses, such as an address bus, a data bus, or any combination thereof. One of skill in the art additionally will appreciate that, depending on the intended applications and uses of a particular embodiment, multiple of these components can be implemented by a single device. Similarly, in some instances, a single component can be implemented by multiple devices. As such, FIG. 17 is merely illustrative of an exemplary computing device that can be used to implement one or more embodiments of the present invention, and in no way limits the invention.

The computing device 1700 can include or interact with a variety of computer-readable media. For example, computer-readable media can include Random Access Memory (RAM); Read Only Memory (ROM); Electronically Erasable Programmable Read Only Memory (EEPROM); flash memory or other memory technologies; CDROM, digital versatile disks (DVD) or other optical or holographic media; magnetic cassettes, magnetic tape, magnetic disk storage or other magnetic storage devices that can be used to encode information and can be accessed by the computing device 1700.

The memory 1712 can include computer-storage media in the form of volatile and/or nonvolatile memory. The memory 1712 may be removable, non-removable, or any combination thereof. Exemplary hardware devices are devices such as hard drives, solid-state memory, optical-disc drives, and the like. The computing device 1700 can include one or more processors that read data from components such as the memory 1712, the various I/O components 1716, etc. Presentation component(s) 1716 present data indications to a user or other device. Exemplary presentation components include a display device, speaker, printing component, vibrating component, etc.

The I/O ports 1718 can enable the computing device 1700 to be logically coupled to other devices, such as I/O components 1720. Some of the I/O components 1720 can be built into the computing device 1700. Examples of such I/O components 1720 include a microphone, joystick, recording device, game pad, satellite dish, scanner, printer, wireless device, networking device, and the like.

As utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may exist in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extent or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the present invention, and exclusive use of all modifications that come within the scope of the appended claims is reserved. Within this specification, embodiments have been described in a way that enables a clear and concise specification to be written, but it is intended and will be appreciated that embodiments may be variously combined or separated without parting from the invention. It is intended that the present invention be limited only to the extent required by the appended claims and the applicable rules of law.

It is also to be understood that the following claims are to cover all generic and specific features of the invention described herein, and all statements of the scope of the invention which, as a matter of language, might be said to fall therebetween.

What is claimed is:

1. A method for providing a personalized nasal dilator, the method comprising:
acquiring data on an individual user's face and/or nasal structure;
analyzing the data to evaluate anatomical and structural needs for the individual user; and
manufacturing a nasal dilator that is personalized for the individual user wherein the nasal dilator is configured, shaped, and dimensioned for the individual user based on the evaluated anatomical and structural needs of the individual user.

2. The method of claim 1, wherein acquiring data on an individual user's face and/or nasal structure comprises using image data to build a three-dimensional (3D) profile of an internal profile of the individual user's nostrils.

3. The method of claim 1, wherein acquiring data on an individual user's face and/or nasal structure comprises using video or photo photogrammetry of one or more of the user's face, exterior of nose and nostrils, and interior of nose and nostrils.

4. The method of claim 1, wherein acquiring data on an individual user's face and/or nasal structure comprises using 3D imaging techniques.

5. The method of claim 4, wherein 3D imaging techniques comprise one or more of: LiDAR, depth or Time of Flight (TOF) cameras, infrared (IR) time of flight, structured infrared (IR) light patterns, and multiple cameras.

6. The method of claim 1, wherein acquiring data on an individual user's face and/or nasal structure comprises using one or more of X-ray, computed tomography (CT) systems, and ultrasound imaging.

7. The method of claim 1, wherein acquiring data on an individual user's face and/or nasal structure comprises using acoustic rhinometry.

8. The method of claim 1, wherein acquiring data on an individual user's face and/or nasal structure further comprises acquiring supplemental posed images or 3D scans of the user comprising one or more of:
an image or 3D scan of the user wrinkling their nose,
an image or 3D scan of the user flaring their nostrils,
an image or 3D scan of the user forcefully inhaling through their nose,
an image or 3D scan where the user holds one nostril closed and an image or 3D scan where the user holds other nostril closed; and
an image or 3D scan where the user holds one nostril open and an image or 3D scan where the user holds other nostril open.

9. The method of claim 1, wherein acquiring data on an individual user's face and/or nasal structure further comprises extracting one or more landmarks.

10. The method of claim 1, wherein acquiring data on an individual user's face and/or nasal structure comprises collecting user profile information.

11. The method of claim 1, wherein analyzing data for the individual user to evaluate anatomical and structural needs for the individual user comprises creating a dilator profile.

12. The method of claim 10, wherein creating a dilator profile comprises a geometric transformation from a 3D Nasal Profile for the user.

13. The method of claim 10, wherein creating a dilator profile comprises creating one or more circumferential radial force profiles.

14. The method of claim 1, wherein analyzing data for the individual user to evaluate anatomical and structural needs for the individual user further comprises:
receiving one or more pro-forma dilator designs, which comprise one or more of: stored templates, one or more stored rules, and one or more stored algorithms; and
modifying the one or more pro-forma dilator designs according to data acquired.

15. The method of claim 1, wherein analyzing data for the individual user provided evaluate anatomical and structural needs for the individual user further comprises:
receiving a 3D Nasal Profile for the user; and
modifying the 3D Nasal Profile for the user using one or more of: one or more stored templates, one or more stored rules, and one or more stored algorithms.

16. The method of claim 1, wherein acquiring data on an individual user's face and/or nasal structure comprises collecting and analyzing data about user response to a nasal dilator.

17. The method of claim 1, wherein manufacturing a nasal dilator that is personalized for the individual user comprises using additive manufacturing methods.

18. The method of claim 1, wherein manufacturing a nasal dilator that is personalized for the individual user comprises:
fabricating a mold for the personalized nasal dilator; and
using the mold to make a personalized nasal dilator.

19. The method of claim 1, wherein manufacturing a nasal dilator that is personalized for the individual user comprises modifying an existing dilator to personalize the dilator for the individual user.

20. A system for providing a personalized nasal dilator; the system comprising:
a data acquisition subsystem configured to receive data on an individual user's facial and nasal structures,
a data transformation subsystem configured to process the data to evaluate nasal characteristics of the individual user; and
a manufacturing subsystem configured to fabricate a customized fitting for the nasal dilator based on the evaluated nasal characteristics.

21. The system of claim 20, wherein the data acquisition subsystem is configured to scan an individual user's face and/or nasal structure to build a three-dimensional (3D) profile of an internal profile of the individual user's nostrils.

22. The system of claim 20, wherein the data acquisition subsystem is configured to use video or photo photogrammetry of one or more of a user's face, exterior of nose and nostrils, and interior of nose and nostrils.

23. The system of claim 20, wherein the data acquisition subsystem is configured to use 3D imaging techniques.

24. The system of claim 20, wherein the data acquisition subsystem is configured to use one or more of X-ray, computed tomography (CT) systems, and ultrasound imaging.

25. The system of claim 20, wherein the data acquisition subsystem is configured to use acoustic rhinometry.

26. The system of claim 20, wherein the data acquisition subsystem is configured to acquire supplemental posed images or 3D scans of the user comprising one or more of:
an image or 3D scan of the user wrinkling their nose,
an image or 3D scan of the user flaring their nostrils,
an image or 3D scan of the user forcefully inhaling through their nose,
an image or 3D scan where the user holds one nostril closed and an image or 3D scan where the user holds another nostril closed; and
an image or 3D scan where the user holds one nostril open and an image or 3D scan where the user holds other nostril open.

27. The system of claim 20, wherein the data acquisition subsystem is configured to extract one or more landmarks.

28. The system of claim 20, wherein the data transformation subsystem is further configured to collect user profile information.

29. The system of claim 20, wherein processing the data to evaluate nasal characteristics of the user comprises creating a dilator profile.

30. The system of claim 20, wherein the data transformation subsystem is configured to:
receive one or more pro-forma dilator designs, which comprise one or more of: stored templates, one or more stored rules, and one or more stored algorithms; and
modify the one or more pro-forma dilator designs according to data provided by the acquisition.

31. The system of claim 20, wherein the data transformation subsystem is configured to:
receive a 3D Nasal Profile for the user; and
modify the 3D Nasal Profile for the user using one or more of: one or more stored templates, one or more stored rules, and one or more stored algorithms.

32. The system of claim 20, wherein the data transformation subsystem is further configured to collect and analyze data about user response to a nasal dilator.

33. The system of claim 20, wherein the manufacturing subsystem uses additive manufacturing methods.

34. The system of claim 20, wherein the manufacturing subsystem is configured to:
fabricate a mold for the personalized nasal dilator; and
use the mold to make a personalized nasal dilator.

35. The system of claim 20, wherein the manufacturing subsystem is configured to modify an existing dilator to personalize the dilator for an individual user.

36. A personalized nasal dilation system, comprising:
one or more nasal inserts structurally adapted to align with a nasal passage surface anatomy of an individual user, each insert comprising one or more of:
one or more varied configurations that fit nasal passage anatomical configurations of an individual user; and
a circumferential force profile configured to vary at different points of contact along a circumference of the insert providing one or more dilation force magnitudes disposed at different locations around the circumference;
wherein the one or more nasal inserts are shaped, sized, and configured based upon anatomical information of nasal passage surface features derived through an analysis of anatomical information for the individual user.

37. The personal nasal dilation system of claim 36, wherein the one or more nasal inserts are formed of a pliant material.

38. The personal nasal dilation system of claim 36, wherein the one or more nasal inserts are comprised of a biocompatible material.

39. The personal nasal dilation system of claim 36, wherein each nasal insert comprises:
a top opening;
a base opening; and
at least one support extending between the top opening and the base opening forming a passage extending between the top opening and the base opening.

40. The personal nasal dilation system of claim 39, wherein the at least one support comprises a wall.

41. The personal nasal dilation system of claim 40, wherein the wall comprises a mesh structure.

42. The personal nasal dilation system of claim 39, wherein the base opening is sized, dimensioned, and configured to engage a vestibular rim of a nose of a user.

43. The personal nasal dilation system of claim 36, wherein the system is configured as part of a CPAP pillow.

44. A personal nasal dilator resulting from:
acquiring data on an individual user's face and/or nasal structure;
analyzing the data to evaluate anatomical and structural needs for the individual user; and
manufacturing a nasal dilator device that is personalized for the individual user wherein the nasal dilator is configured, shaped, and dimensioned for the individual user based on the evaluated anatomical and structural needs of the individual user.

45. A method for providing a personalized nasal dilator, the method comprising:
scanning an individual user's face and/or nasal structure, the scanning comprising:
scanning an individual user's photogrammetry or 3D imaging;
extracting landmarks from the scanning;
analyzing data for the individual user provided by the scanning to evaluate anatomical and structural needs for the individual user, the analyzing comprising:
selecting a pro-forma dilator design based on the landmarks;
modifying the pro-forma dilator design based on the landmarks to address anatomical and structural needs for the individual user; and
generating a dilator profile; and
manufacturing a nasal dilator that is personalized for the individual user wherein the nasal dilator is configured, shaped, and dimensioned for the individual user based on the evaluated anatomical and structural needs of the individual user from the dilator profile.

46. The method of claim 45, wherein analyzing the data further comprises collecting user profile data for the individual user.

47. The method of claim 45, wherein analyzing the data is performed using machine learning.
